(19) United States
(12) Patent Application Publication
(10) Pub. No.: US 2013/0035252 A1
(43) Pub. Date: Feb. 7, 2013

(54) METHODS FOR DETERMINING A GENE EXPRESSION PROFILE AND FOR DISEASE DIAGNOSIS

(71) Applicants: Stanley Letovsky, Milton, MA (US); Stanley Lapidus, Bedford, NH (US)

(72) Inventors: Stanley Letovsky, Milton, MA (US); Stanley Lapidus, Bedford, NH (US)

(73) Assignee: SYNAPDX CORP., Southborough, MA (US)

(21) Appl. No.: 13/566,167

(22) Filed: Aug. 3, 2012

Related U.S. Application Data
(60) Provisional application No. 61/514,620, filed on Aug. 3, 2011.

(51) Int. Cl. C40B 30/04; C12Q 1/68

(52) U.S. Cl. 506/9; 435/6.12

(57) ABSTRACT

The invention generally relates to methods for determining a gene expression profile and for disease diagnosis. In certain aspects, methods of the invention involve obtaining a sample including somatic cells, transforming the somatic cells into target cells, and determining an expression profile from the target cells.

### FIELD OF THE INVENTION

The invention generally relates to methods for determining a gene expression profile and for disease diagnosis.

### BACKGROUND

Gene expression, copy number variation, cellular morphology, mutational analysis and other techniques have proven useful in a variety of ways for assessing the biological status of cells or tissue. For example, gene expression is an indicator of the biology of both normal and abnormal cells. In medical diagnostics, gene expression patterns, such as quantitative RNA, in specific tissues is used to diagnose or classify disease, to assess treatment, and to effect therapeutic choice.

Most celluar and sub-cellular diagnostic tests require obtaining the cells or tissue associated with the target disorder. This presents a problem in situations in which the desired tissue is difficult to obtain. Neurological disorders, in which the affected tissue may be brain tissue, presents an especially difficult problem, as cadaver tissue may be the only readily-available tissue source. Thus, it is very difficult, and often impossible, to obtain cellular or sub-cellular diagnostic information for neural tissue or cells, which limits the ability to perform key diagnostic assays. Therefore, there is a need in the art for methods of obtaining reliable expression profile information from neural tissue.

### SUMMARY

The invention generally provides methods for cellular and/or sub-cellular analysis, of tissue or cell samples without the need to perform a invasive procedures to obtain those samples. Every somatic cell has the same underlying genotype. According to the invention, a somatic cell obtained from a first, accessible tissue source is transformed or transdifferentiated into cells of a second, difficult-to-access tissue source for analysis. The cellular characteristic (e.g., expression profile, copy number, mutation panel, protein complement, cell surface markers, etc.) obtained in the transformed or transdifferentiated cell minors that of the cells in situ.

Methods of the invention are applicable to any tissue that is difficult to obtain. In particular, the invention is useful for transforming or transdifferentiating cells into neuronal cells. The transformed or transdifferentiated cells are analyzed for characteristics of the target cells of origin based upon the underlying equivalent genome. In a preferred method, transformed cells are analyzed for genomic content, and in particular expression patterns indicative of disease status or the potential for disease. In one method, somatic cells are transformed into neuronal cells and analyzed for indicia of autism and autism-related conditions, such as autism spectrum disorder, or other neurological conditions (e.g., dementia, psychiatric disorders and the like). For example, transformed neuronal cells provide an accurate indication of expression characteristics that are then correlated to other clinical indicia. Those correlations are useful in generating a database for further clinical assessment and/or for directly assessing a patient's clinical status. Further, data obtained from the transformed or transdifferentiated cells are useful directly in clinical diagnostic tests.

Any method known in the art may be employed to transform somatic cells into target cells. In certain embodiments, an indirect route that involves dedifferentiation and then redifferentiation of the somatic cells is employed. Such a route involves reprogramming a variety of somatic cell types from different lineages to produce a dedifferentiated embryonic stem cell state. Indirect routes include somatic cell nuclear transfer, cell fusion, or creation of induced pluripotent stem cells by introduction of genes such as Oct4. The dedifferentiated cells are then redifferentiated to target cells along respective mesodermal, endodermal, or ectodermal lineages.

In other embodiments, a direct transdifferentiation route is employed. Such direct transdifferentiation is accomplished by inducing lineage-specific transcription factors encoded by certain genes that result in direct transformation of the somatic cell to the target cell. For example, induction of transcription factors encoded by genes including Ascl1, Brn2, and Myt1l, results in fibroblasts being directly transformed into cortical excitatory neurons.

The somatic cells used in the invention may be any cells in the body. Generally, the somatic cells are cells that can be obtained through a non-invasive or minimally invasive procedure. Such cells include epithelial cells obtained by a cheek swab or a skin punch or white blood cells obtained from drawn blood. The target cells may also be any cells in the body. Generally, the target cells are cells that are difficult to obtain, e.g., cells that can only be obtained by performing a highly invasive surgical procedure.

The target cells are then assayed for a cellular characteristic indicative of a clinical condition. A primary cellular characteristic that is amenable to the invention is a gene expression profile. Any assay known in the art may be used to obtain the gene expression profile. Exemplary assays involve microarray analysis, sequencing, qRTPCR, or similar RNA or cDNA quantitation method.

The obtained gene expression profile may be used to diagnose a particular disorder. Methods of the invention allow for diagnosis of any disorder. In particular, the invention provides methods for identifying indicia of neurological disorders, such as autism spectrum disorder, Bell's Palsy, Parkinson's disease, Creutzeldt-Jakob disease, Hungtington's disease, epilepsy, and aphasia. In particular, the invention provides methods for diagnosing anautism spectrum disorder, such as autism (classical autism), asperger syndrome, rett syndrome, childhood disintegrative disorder, and pervasive developmental disorder not otherwise specified (PDD-NOS).

In certain aspects, the invention provides methods for diagnosing an autism spectrum disorder that involve obtaining a sample including somatic cells, transforming the somatic cells into neurons, and determining an expression profile from the neurons, thereby diagnosing an autism spectrum disorder. The sample may be from a human of any age. In particular embodiments, the sample is from a human child.

Any cellular characteristic may be measured in the transformed or transdifferentiated cells and techniques for doing so are known in the art. See, e.g., Ausubel, et al., Short Protocols in Molecular Biology, Wiley (2002), incorporated by reference herein. For example, DNA copy number variation, mutations (e.g., SNP panels, rearrangements, deletions, etc.), surface proteins, morphological analysis and others are useful diagnostic tools for use on transformed or transdifferentiated cells. The invention also contemplates a database resident on a computer-readable medium comprising markers associated with a disease of interest, wherein at least one of the markers is identified in a transformed or transdifferentiated cell. In a preferred embodiment, the database comprises markers associated with an autism spectrum disorder. While the invention is described in view of the following detailed description, other laboratory techniques are known in the art and are intended to be applied to practice of the invention as described in its broadest terms.

### DETAILED DESCRIPTION

The invention generally relates to methods for determining cellular indicia of disease. In certain aspects, methods of the invention involve obtaining a sample including somatic cells, transforming the somatic cells into target cells, and determining a cellular characteristic of the transdifferentiated cells.

### Samples

Methods of the invention involve obtaining a sample including somatic cells. The sample may be any human tissue or body fluid. Generally, the sample will be a sample that can be obtained through a non-invasive or minimally invasive procedure. For example, the sample may be a sample that includes epithelial cells obtained by a cheek swab or a skin punch or a sample that includes white blood cells obtained from drawn blood. The sample may be collected in any clinically acceptable manner.

Examples of suitable populations of mammalian cells include those that include, but are not limited to: fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, gastric epithelial cells, lung epithelial cells, synovial cells, kidney cells, skin epithelial cells or osteoblasts.

The cells can also originate from many different types of tissue, e.g., bone marrow, skin (e.g., dermis, epidermis), muscle, adipose tissue, peripheral blood, foreskin, skeletal muscle, or smooth muscle. The cells can also be derived from neonatal tissue, including, but not limited to: umbilical cord tissues (e.g., the umbilical cord, cord blood, cord blood vessels), the amnion, the placenta, or other various neonatal tissues (e.g., bone marrow fluid, muscle, adipose tissue, peripheral blood, skin, skeletal muscle etc.).

The cells can be derived from neonatal or post-natal tissue collected from a subject within the period from birth, including cesarean birth, to death. For example, the tissue may be from a subject who is >10 minutes old, >1 hour old, >1 day old, >1 month old, >2 months old, >6 months old, >1 year old, >2 years old, >5 years old, >10 years old, >15 years old, >18 years old, >25 years old, >35 years old, >45 years old, >55 years old, >65 years old, >80 years old, <80 years old, <70 years old, <60 years old, <50 years old, <40 years old, <30 years old, <20 years old or <10 years old. The subject may be a neonatal infant. In some cases, the subject is a child or an adult. In some examples, the tissue is from a human of age 2, 5, 10 or 20 hours. In other examples, the tissue is from a human of age 1 month, 2 months, 3 months, 4 months, 5 months, 6 months, 9 months or 12 months. In some cases, the tissue is from a human of age 1 year, 2 years, 3 years, 4 years, 5 years, 18 years, 20 years, 21 years, 23 years, 24 years, 25 years, 28 years, 29 years, 31 years, 33 years, 34 years, 35 years, 37 years, 38 years, 40 years, 41 years, 42 years, 43 years, 44 years, 47 years, 51 years, 55 years, 61 years, 63 years, 65 years, 70 years, 77 years, or 85 years old.

The cells may be from non-embryonic tissue, e.g., at a stage of development later than the embryonic stage. In other cases, the cells may be derived from an embryo. In some cases, the cells may be from tissue at a stage of development later than the fetal stage. In other cases, the cells may be derived from a fetus.

The cells are preferably from a human subject but can also be derived from non-human subjects, e.g., non-human mammals. Examples of non-human mammals include, but are not limited to, non-human primates (e.g., apes, monkeys, gorillas), rodents (e.g., mice, rats), cows, pigs, sheep, horses, dogs, cats, or rabbits.

The cells may be collected from subjects with a variety of disorder statuses. The cells can be collected from a subject who is free of an adverse health condition. In other cases, the subject is suffering from, or at high risk of suffering from, a disease or disorder, e.g., an autism spectrum disorder.

The cells to be transformed can be obtained from a single cell or a population of cells. The population may be homogeneous or heterogeneous. The cells may be a population of cells found in a human cellular sample, e.g., a biopsy or blood sample. Often, the cells are somatic cells. The cells may be a cell line. In some cases, the cells are derived from cells fused to other cells. In some cases, the cells are not derived from cells artificially fused to other cells. In some cases, the cells are not a cell that has undergone the procedure known as somatic cell nuclear transfer (SCNT) or a cell descended from a cell that underwent SCNT.

Methods for obtaining human somatic cells are well established, as described in, e.g., Schantz and Ng (2004), A Manual for Primary Human Cell Culture, World Scientific Publishing Co., Pte, Ltd., and Sakurada et al. (U.S. patent application number 2009/0191159), the content of each of which is incorporated by reference herein in its entirety. In some cases, the methods include obtaining a cellular sample, e.g., by a biopsy (e.g., a skin sample) or blood draw. It is to be understood that initial plating densities from of cells prepared from a tissue may be varied based on such variable as expected viability or adherence of cells from that particular tissue.

### Transformation of Somatic Cells to Target Cells

Any method known in the art may be employed to transform somatic cells into target cells. In certain embodiments, an indirect route that involves dedifferentiation and then redifferentiation of the somatic cells is employed. Such a route involves reprogramming a variety of somatic cell types from different lineages to produce a dedifferentiated embryonic stem cell state. Indirect routes include somatic cell nuclear transfer, cell fusion, or creation of induced pluripotent stem cells by introduction of genes such as Oct4. The dedifferentiated cells are then redifferentiated to target cells along respective mesodermal, endodermal, or ectodermal lineages. Further description of such methods are found for example in Isacson et al. (U.S. patent application number 2010/0021437), Yamanaka et al. (U.S. patent application number 2009/0047263), Sakurada et al. (U.S. patent application number 2009/0191159), Yamanaka et al. (U.S. patent application number 2009/0227032), Sakurada et al. (U.S. patent application number 2009/0304646), Sakurada et al. (U.S. patent application number 2010/0105100), Takahashi et al. (U.S. patent application number 2010/0105137), Sakurada et al. (U.S. patent application number 2010/0120069), Sakurada et al. (U.S. patent application number 2010/0267135), Hochedlinger et al. (U.S. patent application number 2010/0062534), and Hochedlinger et al. (U.S. patent application number 2010/0184051), the content of each of which is incorporated by reference herein in its entirety. Methods for preparing induced pluripotent stem cells by using a nuclear reprogramming factor are described in International publication number WO 2005/80598, the content of which is incorporated by reference herein in its entirety.

In certain embodiments, transformation involves inducing somatic cells to become pluripotent stem cells or multipotent stem cells, and then differentiating the pluripotent stem cells or multipotent stem cells into target cells. Pluripotent stem cells have the ability to differentiate into cells of all three germ layers (ectoderm, mesoderm and endoderm); in contrast, multipotent stem cells can give rise to one or more cell-types of a particular germ layer(s), but not necessarily all three.

The process of inducing cells to become multipotent or pluripotent is based on forcing the expression of polypeptides, particularly proteins that play a role in maintaining or regulating self-renewal and/or pluripotency of ES cells. Examples of such proteins are the Oct3/4, Sox2, Klf4, and c-Myc transcription factors, all of which are highly expressed in ES cells. Forced expression may include introducing expression vectors encoding polypeptides of interest into cells (Hochedlinger et al., U.S. patent application number 2010/0062534), transduction of cells with recombinant viruses, introducing exogenous purified polypeptides of interest into cells, contacting cells with a non-naturally occurring reagent that induces expression of an endogenous gene encoding a polypeptide of interest (e.g., Oct3/4, Sox2, Klf4, or c-Myc), or any other biological, chemical, or physical means to induce expression of a gene encoding a polypeptide of interest (e.g., an endogenous gene Oct3/4, Sox2, Klf4, or c-Myc). Some basic steps to induce the cells are shown in Sakurada et al. (U.S. patent application number 2009/0191159). These steps may involve: collection of cells from a donor, e.g., a human donor, or a third party; induction of the cells, e.g., by forcing expression of polypeptides such as Oct3/4, Sox2, Klf4, and c-Myc (110); identifying multipotent or pluripotent stem cells; isolating colonies; and optionally, storing the cells. Interspersed between all of these steps are steps to maintain the cells, including culturing or expanding the cells. In addition, storage of the cells can occur after many steps in the process. Cells may later be used in many contexts, such as therapeutics or other uses.

The induced cells may be differentiated into cell-types of various lineages. Examples of differentiated cells include any differentiated cells from ectodermal (e.g., neurons and fibroblasts), mesodermal (e.g., cardiomyocytes), or endodermal (e.g., pancreatic cells) lineages. The differentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, or cardiomyocytes.

The differentiated cells derived from the induced cells may be terminally differentiated cells, or they may be capable of giving rise to cells of a specific lineage. For example, induced cells can be differentiated into a variety of multipotent cell types, e.g., neural stem cells, cardiac stem cells, or hepatic stem cells. The stem cells may then be further differentiated into new cell types, e.g., neural stem cells may be differentiated into neurons; cardiac stem cells may be differentiated into cardiomyocytes; and hepatic stem cells may be differentiated into hepatocytes.

There are numerous methods of differentiating the induced cells into a more specialized cell type, such as those methods described in Sakurada et al. (U.S. patent application number 2009/0191159). Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

In certain embodiments, the cells are differentiated into neural stem cells and further differentiated into neurons. Any known method of generating neural stem cells from ES cells may be used to generate neural stem cells from induced cells. See, e.g., Reubinoff et al., (2001), Nat, Biotechnol., 19(12): 1134-40, the content of which is incorporated by reference herein in its entirety. For example, neural stem cells may be generated by culturing the induced cells as floating aggregates in the presence of noggin, or other bone morphogenetic protein antagonist, see e.g., Itsykson et al., (2005), Mol, Cell Neurosci., 30(1):24-36, the content of which is incorporated by reference herein in its entirety. In another example, neural stem cells may be generated by culturing the induced cells in suspension to form aggregates in the presence of growth factors, e.g., FGF-2, Zhang et al., (2001), Nat. Biotech., (19): 1129-1133, the content of which is incorporated by reference herein in its entirety. In some cases, the aggregates are cultured in serum-free medium containing FGF-2. In another example, the induced cells are co-cultured with a mouse stromal cell line, e.g., PA6 in the presence of serum-free medium comprising FGF-2. In yet another example, the induced cells are directly transferred to serum-free medium containing FGF-2 to directly induce differentiation.

Neural stems derived from the induced cells may be differentiated into neurons, oligodendrocytes, or astrocytes. Often, the conditions used to generate neural stem cells can also be used to generate neurons, oligodendrocytes, or astrocytes.

In order to promote differentiation into dopaminergic neurons, induced cells may be co-cultured with a PA6 mouse stromal cell line under serum-free conditions, see, e.g., Kawasaki et al., (2000) Neuron, 28(1):3140, the content of which is incorporated by reference herein in its entirety. Other methods have also been described, see, e.g., Pomp et al., (2005), Stem Cells 23(7):923-30; U.S. Pat. No. 6,395,546, e.g., Lee et al., (2000), Nature Biotechnol., 18:675-679, the content of each of which is incorporated by reference herein in its entirety.

Oligodendrocytes may also be generated from the induced cells. Differentiation of the induced cells into oligodendrocytes may be accomplished by known methods for differentiating ES cells or neural stem cells into oligodendrocytes. For example, oligodendrocytes may be generated by co-culturing induced cells or neural stem cells with stromal cells, e.g., Hermann et al. (2004), J Cell Sci. 117(Pt 19):4411-22, the content of which is incorporated by reference herein in its entirety. In another example, oligodendrocytes may be generated by culturing the induced cells or neural stem cells in the presence of a fusion protein, in which the Interleukin (IL)-6 receptor, or derivative, is linked to the IL-6 cytokine, or derivative thereof. Oligodendrocytes can also be generated from the induced cells by other methods known in the art, see, e.g. Kang et al., (2007) Stem Cells 25, 419-424, the content of which is incorporated by reference herein in its entirety.

Astrocytes may also be produced from the induced cells. Astrocytes may be generated by culturing induced cells or neural stem cells in the presence of neurogenic medium with bFGF and EGF, see e.g., Brustle et al., (1999), Science, 285:754-756, the content of which is incorporated by reference herein in its entirety.

Other methods for differentiating induced pluripotent stem cells into neural stem cells and further into neurons are described in Isacson et al. (U.S. patent application number 2010/0021437).

In other embodiments, a direct transdifferentiation route is employed. Such direct transdifferentiation is accomplished by inducing lineage-specific transcription factors encoded by certain genes that result in direct transformation of the somatic cell to the target cell. Induction may be accomplished by preparing virus vectors, such as lentivirus vectors, and infecting the somatic cells with the vectors. For example, induction of transcription factors encoded by genes including Ascl1, Brn2, and Myt1l, results in fibroblasts being directly transformed into cortical excitatory neurons. See Vierbuchen et al. (Nature, 463:1035-1041, 2010), the content of which is incorporated by reference herein in its entirety. Further description of methods for directly transforming somatic cells to target cells, particularly fibroblasts to neurons, is shown for example in Vierbuchen et al. (Nature, 463:1035-1041, 2010).

### Expression Analysis

Certain methods of the invention involve determining an expression profile in the transdifferentiated cells. Expression is indicative of clinical status and may be used as a screen indicate the presence of a disorder, such as an autism spectrum disorder. Methods of the invention are based upon the fact that aberrant expression pattern is a function of a patient's underlying genotype. Thus it is possible to reproduce expression characteristics of that genotype by transforming somatic source cells from a more accessible tissue (e.g., epithelial cells from a cheek swab or skin punch) into target cells.

In order to use expression analysis for disorder diagnosis, a threshold of expression is established. The threshold may be established by reference to literature or by using a reference sample from a subject known not to be afflicted with the disorder. The expression profile from the test subject is then compared to the reference threshold. Aberrant expression is an indication that the test subject is afflicted with the disorder. The expression may be over-expression compared to the reference (i.e., an amount greater than the reference) or under-expression compared to the reference (i.e., an amount less than the reference).

Methods of the invention may be used to detect any disorder. In particular embodiments, methods of the invention are used to detect an autism spectrum disorder. For example, if one is testing a child for presence of an autism spectrum disorder, a gene expression profile of the test subject may be compared to a gene expression profile from a subject known not to have an autism spectrum disorder. Aberrant expression (under or over) in the test subject compared to the reference subject indicates that the test subject is afflicted with an autism spectrum disorder.

Methods of detecting levels of gene products (e.g., RNA or protein) are known in the art, and any method known in the art may be used to obtain the expression profile from the target cells. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

A differentially expressed gene or differential gene expression refer to a gene whose expression is activated to a higher or lower level in a subject suffering from a disorder, such as an autism spectrum disorder, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as autism, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step is the isolation of mRNA from a target sample. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). The contents of each of theses references is incorporated by reference herein in their entirety. In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin. For performing analysis on pre-implantation embryos and oocytes, Chuk is a gene that is used for normalization.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g. Held et al., Genome Research 6:986 994 (1996), the contents of which are incorporated by reference herein in their entirety.

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” (Kononen et al., Nat. Med 4(7):844-7 (1998). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484 487 (1995); and Velculescu et al., Cell 88:243 51 (1997, the contents of each of which are incorporated by reference herein in their entirety).

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See for example U.S. Pat. Nos. 6,925,389; 6,989,100; and 6,890,763 for further guidance, each of which is incorporated by reference herein in their entirety.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

1. A method of diagnosing a disease, the method comprising the steps of:
obtaining a sample comprising somatic cells;
dedifferentiating the somatic cells into pluripotent stem cells;
redifferentiating the pluripotent stem cells into target cells; and
identifying a marker in the target cells that is indicative of disease.

2. The method according to claim 1, wherein said marker is expressed RNA.

3. The method according to claim 1, wherein the disease is an autism spectrum disorder.

4. The method according to claim 3, wherein the autism spectrum disorder is selected from the group consisting of autism (classical autism), asperger syndrome, rett syndrome, childhood disintegrative disorder, and pervasive developmental disorder not otherwise specified (PDD-NOS).

5. The method according to claim 1, wherein the target cells are neurons.

6. The method according to claim 1, wherein the somatic cells are selected from the group consisting of skin fibroblasts, epithelial cells and white blood cells.

7. The method according to claim 1, wherein said identifying step comprises conducting an assay selected from the group consisting of: microarray analysis; sequencing; qRTPCR; and a combination thereof.

8. The method according to claim 2, wherein the expressed RNA is compared to a reference RNA expression profile for the disease, thereby diagnosing the disease.

9. The method according to claim 1, further comprising the step of comparing said marker to a database comprising markers known to be associated with an autism spectrum disorder.

10. The method according to claim 9, further comprising the step of creating a database of markers known to be associated with an autism spectrum disorder.
